(19) United States
(12) Patent Application Publication
(10) Pub. No.: US 2003/0144518 A1
(43) Pub. Date: Jul. 31, 2003

(54) Process for preparing 3-acylamino-imidazo[1,2-a]pyridines

(75) Inventor: Jian Chen, Mason, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/302,246

(22) Filed: Nov. 22, 2002

Related U.S. Application Data
(60) Provisional application No. 60/340,497, filed on Dec. 7, 2001.

(51) Int. Cl. C07D 471/02

(52) U.S. Cl. 546/121

(57) ABSTRACT

The present invention relates to a process for preparing 3-acylamino imidazo[1,2-a]pyridines having the formula:

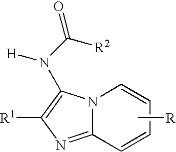

wherein R is one or more units which can substitute for hydrogen ring atoms; R¹ is C₁-C₁₂ linear, branched, or cyclic alkyl; C₆-C₁₀ substituted or unsubstituted aryl; C₇-C₁₂ substituted or unsubstituted alkylenearyl; R² is C₁-C₁₂ linear, branched, or cyclic alkyl; C₆-C₁₀ substituted or unsubstituted aryl; C₇-C₁₂ substituted or unsubstituted alkylenearyl; C₂-C₁₀ substituted or unsubstituted heteroaryl; said process comprising the steps of:

a) reacting an isonitrile resin with an aldehyde having the formula R¹CHO and a substituted or unsubstituted 2-aminopyridine in the presence of an acid catalyst to form a resin bound 2-substituted-3-aminoimidazo[1 ,2-a]pyridine; and

b) cleaving said resin bound 2-substituted-3-aminoimidazo[1,2-a]pyridine substrate from said resin by reacting said substrate with an acyl halide having the formula R²COX; X is chlorine or bromine, to form a said 3-acylamino imidazo[1,2-a]pyridine.

### CROSS REFERENCE TO RELATED APPLICATIONS

This application claims priority under Title 35, United States Code 119(e) from Provisional Application Serial No. 60/340,497, filed Dec. 7, 2001.

### FIELD OF THE INVENTION

The present invention relates to a process for preparing 3-acylaminoimidazo[1,2-a]pyridines. The present invention thereby also provides a facile synthesis of 3-aminoimidazo[1,2-a]pyridines.

### BACKGROUND OF THE INVENTION

3-Acylaminoimidazo[1,2-a]pyridines and 3-aminoimidazo[1,2-a]pyridines are important scaffolds for the preparation of pharmaceuticals, pesticides, and other biologically active ingredients. Multi-component reactions (MCR's), inter alia, Ugi and Passerini reactions have been well described in the literature, however, these reactions suffer from the same work-up and isolation constrains as other non-MCR synthetic procedures.

There is therefore a long felt need to utilize multi-component reactions in a manner wherein the isolation of reaction intermediates is facilitated, especially in the case wherein low yield transformations are anticipated or realized.

### SUMMARY OF THE INVENTION

The present invention addresses the aforementioned needs in that herein is provided a facile means for preparing 3-acylaminoimidazo[1,2-a]pyridines which are also pre-cursors to the broader class of 3-aminoimidazo[1,2-a]pyridines. It has now been surprisingly discovered that the amino group of amine comprising resins, inter alia, Rink resin can be modified to an isonitrile unit and this unit can then participate in any of a number of MCR reactions involving substituted or unsubstituted 2-aminopyridines and selected aldehydes to form a resin bound substrate wherein the aldehyde substituent occupies the 2-position of the 3-aminoimidazo[1,2-a]pyridine scaffold.

It has also been surprisingly discovered that cleavage of the 3-aminoimidazo[1,2-a]pyridine scaffold from the resin can be accomplished under mild conditions which allow the formulator entry into the 3-acylaminoimidazo[1,2-a]pyridine class of compounds.

The first aspect of the present invention relates to a process for preparing 3-acylamino imidazo[1,2-a]pyridines having the formula:

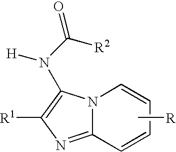

wherein R is one or more units which can substitute for hydrogen ring atoms; R¹ is C₁-C₁₂ linear, branched, or cyclic alkyl; C₆-C₁₀ substituted or unsubstituted aryl; C₇-C₁₂ substituted or unsubstituted alkylenearyl; R² is C₁-C₁₂ linear, branched, or cyclic alkyl; C₆-C₁₀ substituted or unsubstituted aryl; C₇-C₁₂ substituted or unsubstituted alkylenearyl; C₂-C₁₀ substituted or unsubstituted heteroaryl; said process comprising the steps of:

a) reacting an isonitrile resin with an aldehyde having the formula R¹CHO and a substituted or unsubstituted 2-aminopyridine in the presence of an acid catalyst to form a resin bound 2-substituted-3-aminoimidazo[1,2-a]pyridine; and

b) cleaving said resin bound 2-substituted-3-aminoimidazo[1,2-a]pyridine substrate from said resin by reacting said substrate with an acyl halide having the formula R²COX; X is chlorine or bromine, to form a said 3-acylamino imidazo[1,2-a]pyridine. The second aspect of the present invention relates to a process for preparing 3-acylamino imidazo[1,2-a]pyridines having the formula:

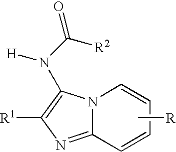

wherein R is one or more units which can substitute for hydrogen ring atoms; R¹ is C₁-C₁₂ linear, branched, or cyclic alkyl; C₆-C₁₀ substituted or unsubstituted aryl; C₇-C₁₂ substituted or unsubstituted alkylenearyl; R² is C₁-C₁₂ linear, branched, or cyclic alkyl; C₆-C₁₀ substituted or unsubstituted aryl; C₇-C₁₂ substituted or unsubstituted alkylenearyl; C₂-C₁₀ substituted or unsubstituted heteroaryl; said process comprising the steps of:

a) reacting an amine comprising resin with formaldehyde in the presence of a base to form an N-formylated resin;

b) reacting said N-formylated resin with a dehydrating agent to form an isonitrile resin;

c) reacting said isonitrile resin with an aldehyde having the formula R¹CHO and a substituted or unsubstituted 2-aminopyridine in the presence of an acid catalyst to form a resin bound 2-substituted-3-aminoimidazo[1,2-a]pyridine; and

d) cleaving said resin bound 2-substituted-3-aminoimidazo[1,2-a]pyridine substrate from said resin by reacting said substrate with an acyl halide having the formula R²COX; X is chlorine or bromine, to form a said 3-acylamino imidazo[1,2-a]pyridine. The third aspect of the present invention relates to a process for preparing 3-acylamino imidazo[1,2-a]pyridines having the formula:

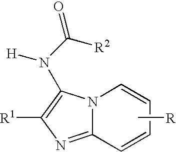

wherein R is one or more units which can substitute for hydrogen ring atoms; R¹ is C₁-C₁₂ linear, branched, or cyclic alkyl; C₆-C₁₀ substituted or unsubstituted aryl; C₇-C₁₂ substituted or unsubstituted alkylenearyl; R² is C₁-C₁₂ linear, branched, or cyclic alkyl; C₆-C₁₀ substituted or unsubstituted aryl; C₇-C₁₂ substituted or unsubstituted alkylenearyl; C₂-C₁₀ substituted or unsubstituted heteroaryl; said process comprising the steps of:

a) converting Rink resin, said resin having the formula:

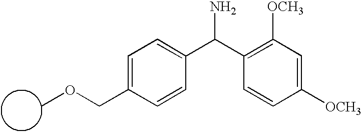

to an isonitrile resin having the formula:

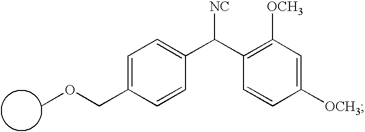

b) reacting said isonitrile resin with an aldehyde having the formula R¹CHO and a substituted or unsubstituted 2-aminopyridine having the formula:

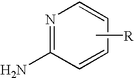

to form a resin bound 2-substituted-3-aminoimidazo[1,2-a]pyridine having the formula:

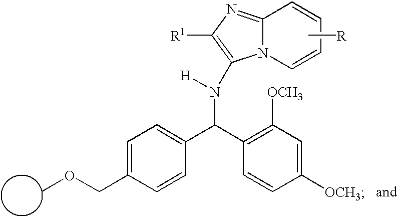

c) reacting said 2-substituted-3-aminoimidazo[1,2-a]pyridine with an acyl chloride having the formula R²COCl to form said 3-acylamino imidazo[1 ,2-a]pyridine.

These and other objects, features, and advantages will become apparent to those of ordinary skill in the art from a reading of the following detailed description and the appended claims. All percentages, ratios and proportions herein are by weight, unless otherwise specified. All temperatures are in degrees Celsius (° C.) unless otherwise specified. All documents cited are in relevant part, incorporated herein by reference; the citation of any document is not to be construed as an admission that it is prior art with respect to the present invention.

### DETAILED DESCRIPTION OF THE INVENTION

The present invention relates to the resin bound preparation of 3-acylaminoimidazo[1,2-a]pyridines which can also serve as direct pre-cursors to the broader, 3-aminoimidazo[1,2-a]pyridines. The process of the present invention encompasses modifying a primary amino unit comprising resin, for example, Rink resin to an isonitrile resin which can then participate in a multi-component reaction to form a compound having the 3-aminoimidazo[1,2-a]pyridine scaffold which is bound to the resin. The scaffold can then be liberated by reaction of the bound substrate with an acid halide which results in the scaffold being isolated as a 3-acylaminoimidazo[1,2-a]pyridine.

For the purposes of the present invention the term “isonitrile” and “isocyanide” are taken to mean the organic functional unit having the formula:

—NC

and these two terms stand equally well for each other and are used interchangeably herein to mean the same functional unit.

For the purposes of the present invention the term “isonitrile resin” or “isocyanide resin” is defined herein as “a resin comprising one or more isonitrile or isocyanide moieties which are capable of reacting with an aldehyde and a substituted or unsubstituted 2-aminopyridine to form a resin bound 3-acylaminoim idazo[1 ,2-a]pyridine.”

The term “substituted” means that any hydrogen atom bonded to a carbon can be replaced by another unit which is chemically compatible with the conditions of the process described herein below. Non-limiting examples of units which can substitute for hydrogen include linear, branched, and cyclic alkyl, halogen, and the like.

### PROCESS

Step (a)

In the broadest and first aspect, Step (a) of the process of the present invention is a multi-component reaction step, MCR step, which encompasses reacting an isonitrile resin with an aldehyde having the formula R¹CHO wherein R¹ is C₁-C₁₂ linear, branched, or cyclic alkyl; C₆-C₁₀ substituted or unsubstituted aryl; C₇-C₁₂ substituted or unsubstituted alkylenearyl; and a substituted or unsubstituted 2-aminopyridine in the presence of an acid catalyst to form a resin bound 2-substituted-3-aminoimidazo[1,2-a]pyridine. The substituted 2-aminopyridine can be represented by the formula:

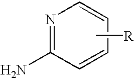

wherein R represent one or more units which substitute for hydrogen. The units which can substitute for hydrogen are those which are typically un-reactive to the conditions of the present process, for example, alkyl, halogen, nitrile, nitro, and the like.

Step (a) can be conducted in the presence of any one of many non-reactive organic solvents, non-limiting examples of which includes dichloromethane, chloroform, ethyl acetate, dimethylsulfoxide, N,N-dimethylformamide, and the like.

The second aspect of Step (a) of the process of the present invention relates to the two steps necessary for forming an isonitrile resin. These steps, Step(a.1) and Step (a.2), when taken together form the nitrile resin of Step (a). Step (a.1) comprises N-formylating the amino units of an amine-containing resin and Step (a.2) comprises dehydrating the N-formyl unit to an isonitrile unit. Step (a.2) may be accomplished without isolation of the intermediate formylated amine, and therefore, in a first iteration of the second aspect the present invention these two steps can be considered as a single step. Likewise, the resin-bound product formed in Step (a.1) can be isolated and purified prior to dehydration thereby resulting in a resin bound N-formyl amine-unit comprising resin (isonitrile precursor). The second iteration of this second aspect of Step (a) treats both Step (a.1) and Step(a.2) as separate discrete steps.

Step (a.1), the preparation of the isonitrile resin, can be accomplished by formylating an amine-comprising resin with formic acid in the presence of a catalyst. For example, Rink resin is reacted with formic acid in the presence of a coupling reagent. The coupling reagent can be any of the coupling reagents which are common to resin bound peptide synthesis. Non-limiting examples of which include diisopropylcarbodiimide (DIC) and dicyclodhexylcarbodiimide (DCC). However, the formulator may N-formylate the amino unit comprising resin by any non-resin destructive procedure including acid catalyzed formylation.

Step (a. 1) may be conducted in the presence of any one of many non-reactive organic solvents, non-limiting examples of which includes dichloromethane, chloroform, ethyl acetate, dimethylsulfoxide, N,N-dimethylformamide, and the like.

Step (a.1) may be conducted at any temperature which is necessary for completion of isonitrile resin formation. Typically the formylation reaction is conducted at 0° C. and allowed to warm to room temperature after the reagents are combined.

Step (a.2) relates to dehydration of the N-formyl amine. This step can be accomplished in the same vessel with out purification or the resin can be rinsed of reactants which are left over from the formulation step.

Suitable dehydration agents include phosphorousoxychloride (POCl₃) which is used in the presence of a base to absorb the hydrogen chloride which is formed as a reaction by product. However, the formulator may utilize any dehydrating agent compatible with the organic based resin.

Step (a.2) may be conducted at any temperature which is necessary for completion of isonitrile resin formation. Typically the dehydration reaction is conducted at 0° C. and allowed to warm to room temperature after the reagents are combined.

However, the discrete steps of the present process do not necessarily need to be accomplished at the same time. For example, the formulator may prepare the isonitrile resin in quantity and use discrete amounts for multiple reactions, especially when conducting MCR's in a combinatorial library mode. In this case, Step (a) can be regarded as Step (a′) which consists of:

a′) providing an isonitrile resin, for example, an isonitrile resin derived from Rink resin having the formula:

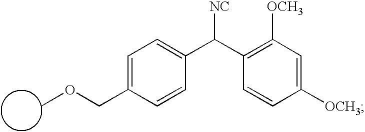

which is then followed by the first aspect of Step (a):

a) reacting an isonitrile resin with an aldehyde having the formula R¹CHO and a substituted or unsubstituted 2-aminopyridine in the presence of an acid catalyst to form a resin bound 2-substituted-3-aminoimidazo[1,2-a]pyridine.

Whenever Step (a.1) and Step (a.2) are combined together is a step which converts an amine-comprising resin to an isonitrile, it is referred to herein as Step (a′).

Step (a) of the present invention can stand alone without Steps (a.1) and (a.2) since, as described herein above, the formulator can stockpile suitable isonitrile resin.

For the purposes of the present invention, Step (a′) can serve as an explicit step especially when the present process is coupled with other synthetic steps. For example, the previous steps of a process may involve the preparation of a particular aldehyde wherein without isolation or purification of said aldehyde, an isonitrile resin is provided into the crude reaction mixture together with a 2-aminopyridine and the admixture reacted together to form a resin bound resin bound 2-substituted-3-aminoimidazo[1,2-a]pyridine.

Step (b)

Step (b) according to the first aspect of the present invention relates to reacting a resin bound 2-substituted-3-aminoimidazo[1,2-a]pyridine with an acyl halide having the formula R²COX, X is chlorine or bromine, to form said 3-acylamino imidazo[1 ,2-a]pyridine. The acyl halide may be any acyl halide wherein R² is C₁-C₁₂ linear, branched, or cyclic alkyl; C₆-C₁₀ substituted or unsubstituted aryl; C₇-C₁₂ substituted or unsubstituted alkylenearyl; C₂-C₁₀ substituted or unsubstituted heteroaryl. Acyl chlorides are typically more easily prepared, more reactive, and most convenient to use, however, acyl bromides are also suitable for use. Non-limiting examples of acyl chlorides include those selected from the group consisting of benzoyl chloride, 3-chlorobenzoyl chloride, furan-2-carbonyl chloride, cyclopropanoyl chloride, and mixtures thereof.

It is understood by formulators who utilize reactive resins for organic chemical synthesis will typically swell the resin using an organic solvent, inter alia, methylene chloride. This and other common practices of the artisan are encompassed within the processs of the present invention.

The following is an example of the process of the present invention.

### EXAMPLE 1

Preparation of N-(2-phenethyl-imidazo[1,2-a]pyridin-3-yl-benzamide: Hydrocinnam-aldehyde (3.08 mL, 14.4 mmol), 2-aminopyridine (1.36 g, 14.4 mmol), para-toluenesulfonic acid (0.32 g, 1.8 mmol) and an admixture of chloroform/methanol/trimethylorthoformate (1:1:1) are combined and the mixture stirred for 2 hours at room temperature. This solution is then added to Rink resin which has been modified to an isonitrile resin (2.0 g, 1.8 mmol) and the suspension agitated overnight. The resulting resin containing the bound 2-phenethyl-3-amino-imidazo[1,2-1]pyridine is washed with N,N-dimethylformamide, methanol, 10% diisopropylethylamine in methylenechloride, methylenechloride, methanol, then dried.

The resin is then suspended in dichloroethane and benzoyl chloride (0.17 mL, 0.96 mmol) and agitated at room temperature for 5 hours after which it is agitated at 50° C. for 16 hours. The contents of the flask is filtered and the filtrate collected and concentrated in vacuo to give an oily residue which is taken up in methanol and added DOWEX4-500 ion exchange resin (1.0 g) is added. The suspension is agitated for 5 hours at room temperature after which the resin is filtered, washed with N,N-dimethylformamide, methanol, methylenechloride, and methanol. The DOWEX resin is then treated with 10% diisopropylethylamine in methanol, the filtrate collected, concentrated in vacuo, and purified by prep HPLC to afford 24.3 mg (31 % yield) of the desired product. ¹H NMR (300 MHz, CD₃OD, δ) 8.53 (dt, J₁=1.1 Hz, J₂=2.0 Hz, J₃=6.8 Hz, 1H), 8.10 (m, 1H), 8.019 (m, 1H), 7.93 (m, 1H), 7.73 (m, 1H), 7.63 (m, 1H), 7.53 (m, 1H), 7.29-7.19 (m, 4H), 3.24 (m, 2H), 3.11 (m, 2H). ¹³C{¹H} (150MHz, CD₃OD, δ) 26.1, 33.9, 111.8, 117.2, 118.6, 125.7, 126.5, 128.1, 128.2, 128.6, 128.8, 132.1, 133.1, 133.7, 138.0, 140.0, 168.7. MS (DCI): m/z 342 [M+H]⁺.

### Preparation of Isonitrile Resin

An amino group comprising resin is suspended in methylene chloride, cooled to 0° C. and pyridine (1 eq.), HCO₂H (5 eq.) and diisopropylcarbodiimide (5 eq.) are added. The suspension of resin is stirred one hour in the cold then about 3 hours at room temperature. At this point the resin can be washed with solvent (N,N-dimethylformamide, methanol, then methylene chloride), dried and the resulting N-formylated resin stored. Or alternatively the next step can be conducted.

The N-formylated resin is suspended in methylene chloride, cooled to 0° C. and diisopropylethylamine (15 eq.) is added. The cautiously, POCl₃ (5 eq.) is added and the suspension stirred in the cold for about 5 hours then allowed to warm and stir at room temperature about 1 hour. The resin is collected by filtration then dried to a constant weight.

Table I provides non-limiting examples of compounds which can be prepared by the process of the present invention.

TABLE I

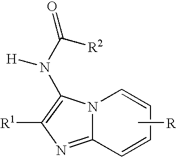

| No. | aminopyridine reagent | R¹ | R² | % yield |
|---|---|---|---|---|
| 1 | 2-aminopyridine | 2-phenylethyl | phenyl | 30.8 |
| 2 | 2-aminopyridine | 2-phenylethyl | 3-chlorophenyl | 33.7 |
| 3 | 2-aminopyridine | 2-phenylethyl | furan-2-yl | 23.4 |
| 4 | 2-aminopyridine | 2-phenylethyl | cyclopropyl | 30.4 |
| 5 | 2-aminopyridine | phenyl | phenyl | 58.0 |
| 6 | 2-aminopyridine | phenyl | furan-2-yl | 60.3 |
| 7 | 2-aminopyridine | phenyl | cyclopropyl | 64.1 |
| 8 | 2-amino-5-methyl- | 3-chlorophenyl | 3-chlorophenyl | 37.7 |
| | pyridine | | | |
| 9 | 2-amino-5-methyl- | 4-chlorophenyl | cyclopropyl | 45.9 |
| | pyridine | | | |
| 10 | 2-amino-5-methyl- | 3-nitrophenyl | 3-methoxy- | 21.6 |
| | pyridine | | benzyl | |
| 11 | 2-amino-5-methyl- | tert-butyl | 3-chlorophenyl | 28.2 |
| | pyridine | | | |
| 12 | 2-amino-3-benzoxy- | furan-2-yl | 3-chlorophenyl | 42.6 |
| | pyridine | | | |
| 13 | 2-amino-3-benzoxy- | 3-methoxy- | cyclopropyl | 66.5 |
| | pyridine | benzyl | | |
| 14 | 2-amino-3-benzoxy- | 2-bromophenyl | 3-methoxy- | 23.8 |
| | pyridine | | benzyl | |
| 15 | 2-amino-3-benzoxy- | cyclohexyl | 3-chlorophenyl | 27.7 |
| | pyridine | | | |
| 16 | 2-amino-3-benzoxy- | cyclopropyl | cyclopropyl | 82.8 |
| | pyridine | | | |
| 17 | 2-amino-3-benzoxy- | isobutyl | 3-methoxy- | 37.8 |
| | pyridine | | benzyl | |

While particular embodiments of the present invention have been illustrated and bed, it would be obvious to those skilled in the art that various other changes and cations can be made without departing from the spirit and scope of the invention. It is ore intended to cover in the appended claims all such changes and modifications that are the scope of this invention.

What is claimed is:

1. A process for preparing 3-acylamino imidazo[1,2-a]pyridines having the formula: 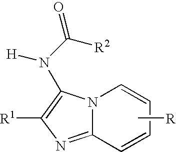
wherein R is one or more units which can substitute for hydrogen ring atoms; R¹ is C₁-C₁₂ linear, branched, or cyclic alkyl; C₆-C₁₀ substituted or unsubstituted aryl; C₇-C₁₂ substituted or unsubstituted alkylenearyl; R² is C₁-C₁₂ linear, branched, or cyclic alkyl; C₆-C₁₀ substituted or unsubstituted aryl; C₇-C₁₂ substituted or unsubstituted alkylenearyl; C₂-C₁₀ substituted or unsubstituted heteroaryl; said process comprising the steps of:
a) reacting an isonitrile resin with an aldehyde having the formula R¹CHO and a substituted or unsubstituted 2-aminopyridine in the presence of an acid catalyst to form a resin bound 2-substituted-3-aminoimidazo[1,2-a]pyridine; and
b) cleaving said resin bound 2-substituted-3-aminoimidazo[1,2-a]pyridine substrate from said resin by reacting said substrate with an acyl halide having the formula R²COX; X is chlorine or bromine, to form a said 3-acylamino imidazo[1,2-a]pyridine.

2. A process for preparing 3-acylamino imidazo[1,2-a]pyridines having the formula: 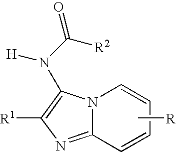
wherein R is one or more units which can substitute for hydrogen ring atoms; R¹ is C₁-C₁₂ linear, branched, or cyclic alkyl; C₆-C₁₀ substituted or unsubstituted aryl; C₇-C₁₂ substituted or unsubstituted alkylenearyl; R² is C₁-C₁₂ linear, branched, or cyclic alkyl; C₆-C₁₀ substituted or unsubstituted aryl; C₇-C₁₂ substituted or unsubstituted alkylenearyl; C₂-C₁₀ substituted or unsubstituted heteroaryl; said process comprising the steps of:
a.1) reacting an amine comprising resin with formaldehyde in the presence of a base to form an N-formylated resin;
a.2) reacting said N-formylated resin with a dehydrating agent to form an isonitrile resin;
b) reacting said isonitrile resin with an aldehyde having the formula R¹CHO and a substituted or unsubstituted 2-aminopyridine in the presence of an acid catalyst to form a resin bound 2-substituted-3-aminoimidazo[1,2-a]pyridine; and
c) cleaving said resin bound 2-substituted-3-aminoimidazo[1,2-a]pyridine substrate from said resin by reacting said substrate with an acyl halide having the formula R²COX; X is chlorine or bromine, to form said 3-acylamino imidazo[1 ,2-a]pyridine.

3. A process for preparing 3-acylamino imidazo[1,2-a]pyridines having the formula: 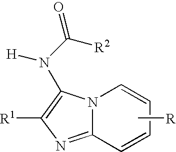
wherein R is one or more units which can substitute for hydrogen ring atoms; R¹ is C₁-C₁₂ linear, branched, or cyclic alkyl; C₆-C₁₀ substituted or unsubstituted aryl; C₇-C₁₂ substituted or unsubstituted alkylenearyl; R² is C₁-C₁₂ linear, branched, or cyclic alkyl; C₆-C₁₀ substituted or unsubstituted aryl; C₇-C₁₂ substituted or unsubstituted alkylenearyl; C₂-C₁₀ substituted or unsubstituted heteroaryl; said process comprising the steps of:
a.1) reacting Rink resin, said resin having the formula: 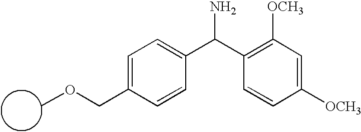
with formaldehyde to form an N-formylated resin having the formula: 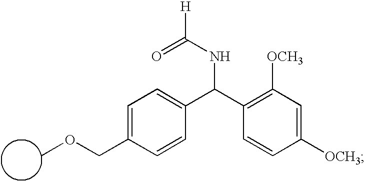
a.2) dehydrating said N-formylated resin with a dehydrating agent to form an isonitrile resin having the formula: 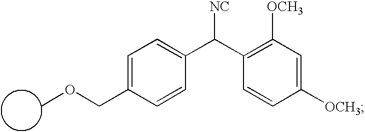
a) reacting said isonitrile resin with an aldehyde having the formula R¹CHO and a substituted or unsubstituted 2-aminopyridine having the formula: 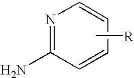
to form a resin bound 2-substituted-3-aminoimidazo[1,2-a]pyridine having the formula: 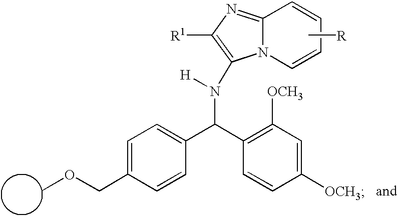
b) reacting said 2-substituted-3-aminoimidazo[1 ,2-a]pyridine with an acyl chloride having the formula R²COCl to form said 3-acylamino imidazo[1,2-a]pyridine.

4. A process according to claim 3 wherein R¹ is selected from the group consisting of phenyl, benzyl, and 2-phenylethyl.

5. A process according to claim 3 wherein R² is C₆-C₁₀ substituted or unsubstituted aryl.

6. A process according to claim 3 wherein R² is C₂-C₁₀ substituted or unsubstituted heteroaryl.

7. A process according to claim 3 wherein R² is C₃-C₁₂ substituted or unsubstituted carbocyclic.

8. A process according to claim 3 wherein said substituted or unsubstituted 2-aminopyridine is selected from the group consisting of 2-aminopyridine, 3-methyl-2-aminopyridine, 4-methyl-2-aminopyridine, 5-methyl-2-aminopyridine, 4-phenyl-2-aminopyridine, and 5-phenyl-2-aminopyridine.

9. A process according to claim 3 wherein said substituted or unsubstituted 2-aminopyridine is 2-aminopyridine.

10. A process according to claim 3 wherein said Rink resin in step (a.1) is converted to said isonitrile resin by reacting said Rink resin with formic acid in the presence of a coupling reagent to form a formylated Rink resin and reacting said formylated Rink resin with a dehydrating agent to form said isonitrile resin.

11. A process according to claim 10 wherein said Rink resin is formylated by contacting said resin with formic acid in the presence of a base.

12. A process according to claim 11 wherein said base is selected from the group consisting of pyridine, pyrimidine, collidine, triethylamine, diisopropylmethylamine, and mixtures thereof.

13. A process according to claim 10 wherein said catalyst is phosphorousoxychloride.

14. A process according to claim 3 wherein step (a) is conducted in the presence of a catalyst.

15. A process according to claim 14 wherein said catalyst is selected from the group consisting of benzenesulfonic acid, toluenesulfonic acid, cumenesulfonic acid, and mixtures thereof.

16. A process according to claim 3 wherein said acyl chloride in step (b) is selected from the group consisting of benzoyl chloride, 3-chlorobenzoyl chloride, furan-2-carbonyl chloride, cyclopropanoyl chloride, and mixtures thereof.

17. A process for preparing 3-acylamino imidazo[1,2-a]pyridines having the formula: 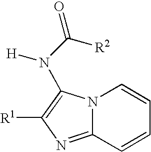
wherein R¹ is selected from the group consisting of substituted or unsubstituted phenyl, benzyl, and 2-phenylethyl; R² is selected from the group consisting of phenyl, furan-2-yl, and cyclopropyl; said process comprising the steps of:
a) reacting Rink resin, said resin having the formula: 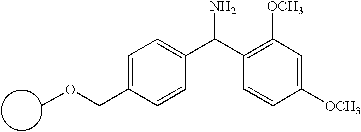
with formaldehyde in the presence of a base to form a formylated Rink resin having the formula: 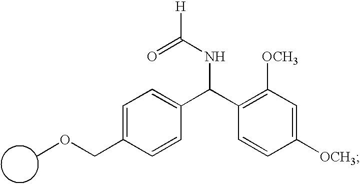
b) reacting said formylated Rink resin with phosophorousoxychloride in the presence of a base to form an isonitrile resin having the formula: 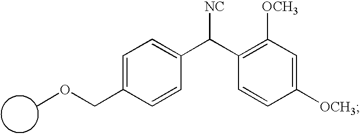
c) reacting said isonitrile resin with an aldehyde having the formula R¹CHO and 2-aminopyridine in the presence of an acid catalyst to form a resin bound 2-substituted-3-aminoimidazo[1,2-a]pyridine substrate having the formula: 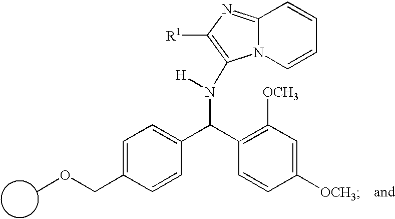
d) cleaving said resin bound 2-substituted-3-aminoimidazo[1,2-a]pyridine substrate from said resin by reacting saidsubstrate with an acyl chloride selected from the group consisting of benzoyl chloride, 3-chlorobenzoyl chloride, furan-2-carbonyl chloride, and cyclopropanoyl chloride to form said 3-acylamino imidazo[1,2-a]pyridine.

18. A process according to claim 17 wherein said acid catalyst in step (c) is selected from the group consisting of benzenesulfonic acid, toluenesulfonic acid, cumenesulfonic acid, and mixtures thereof.

19. A process according to claim 17 wherein said base I step (b) is selected from the group consisting of pyridine, pyrimidine, collidine, triethylamine, diisopropylmethylamine, and mixtures thereof.

20. A process for preparing 3-acylamino imidazo[1,2-a]pyridines having the formula: 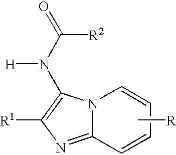
wherein R is one or more units which can substitute for hydrogen ring atoms; R¹ is C₁-C₁₂ linear, branched, or cyclic alkyl; C₆-C₁₀ substituted or unsubstituted aryl; C₇-C₁₂ substituted or unsubstituted alkylenearyl; R² is C₁-C₁₂ linear, branched, or cyclic alkyl; C₆-C₁₀ substituted or unsubstituted aryl; C₇-C₁₂ substituted or unsubstituted alkylenearyl; C₂-C₁₀ substituted or unsubstituted heteroaryl; said process comprising the steps of:
a) reacting an isonitrile resin formed from Rink resin with an aldehyde having the formula R¹CHO and a substituted or unsubstituted 2-aminopyridine having the formula: 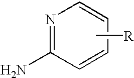
to form a resin bound 2-substituted-3-aminoimidazo[1,2-a]pyridine having the formula: 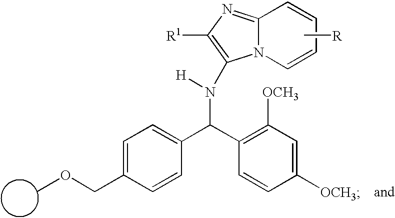
c) reacting said 2-substituted-3-aminoimidazo[1,2-a]pyridine with an acyl chloride having the formula R²COCl to form said 3-acylamino imidazo[1,2-a]pyridine.
